(19) United States
(12) Patent Application Publication
(10) Pub. No.: US 2010/0101587 A1
(43) Pub. Date: Apr. 29, 2010

(54) COSMETIC PROSTHESIS AND METHODS FOR MAKING THE SAME

(76) Inventor: Craig Bruce, Encinitas, CA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/651,776

(22) Filed: Jan. 4, 2010

Related U.S. Application Data
(63) Continuation of application No. 12/013,081, filed on Jan. 11, 2008, now abandoned.
Application No. 12/013,081 is a continuation of application No. 10/539,433, filed on Oct. 20, 2005, now abandoned.
Application No. 10/539,433 is a continuation of application No. 10/346,946, filed on Jan. 17, 2003, now abandoned.
(60) Provisional application No. 60/435,031, filed on Dec. 19, 2002.

(51) Int. Cl. A45D 44/22; A61B 19/00

(52) U.S. Cl. 128/899

(57) ABSTRACT

A cosmetic prosthesis designed to provide non-invasive, removable enhancement of upper and lower lip, being intended to contour and push the facial skin around the mouth and jaw outward, reducing the appearance of wrinkles or aging lines.

### CROSS-REFERENCE TO RELATED APPLICATIONS

The application is a continuation of U.S. application Ser. No. 12/013,081, filed Jan. 11, 2008, which is a continuation of Ser. No. 10/539,433 filed Oct. 20, 2005, which is a National Stage of PCT/US03/40989 filed Dec. 18, 2003, which is a continuation of Ser. No. 10/346,946 filed Jan. 17, 2003, which claims benefit of 60/435,031 filed Dec. 19, 2002, the disclosures of which are incorporated herein by reference in their entirety.

### FIELD OF THE INVENTION

The invention relates generally to cosmetic devices for improving a person's appearance. More specifically, the invention relates cosmetic devices for improving the appearance of the face.

### BACKGROUND OF THE INVENTION

Since the dawn of civilization or since ancient Greece, people have searched for the ‘fountain of youth’ or a manner in which to turn back the hands of time and appear more youthful. As we age, lines and wrinkles become more numerous and prominent, particularly in the face causing additional and pronounced wrinkling of the mouth area. The area of skin surrounding ones mouth is highly susceptible to such wrinkles and aging lines due to the amount of fat cells present in the area and the continual growth of the nose and chin. As we age our teeth and gums also begin to recede, causing additional wrinkling to the mouth and lip area. Smoking, sun exposure and other environmental conditions also add to the aging and wrinkling of the face.

During the aging process, the nose continues to grow putting continual pressure on the upper lip causing ones teeth to show less and less during conversation and smiling. A need exists for a device capable of restoring the upper lip position allowing a more youthful appearance of the lower face addressing the upper and lower lip area. The prosthesis width or thickness will determine the amount of lip lift achieved.

As we age, our teeth and gums also begin to recede into the mouth. This movement of the teeth causes additional wrinkling around the mouth area as the skin folds sag without support, a condition which is exaggerated with the loss of skin elasticity, a condition that inevitably accompanies aging.

Ageing can also lead to a downward turn of the corners of the mouth (frown) that occurs when facial connective tissues and muscles (especially the Zygomaticus muscles, and the Levator Anguli Oris muscle) loosen with age.

A need exists to improve additional appearance conditions:

Lipstick Lines (peri-oral rhytids)

Bell's (or Facial Nerve) palsy

Long upper lips that obscure the upper teeth (i.e. can slightly elevate upper lip)

Hemi facial microsomia: This is when one half of the face doesn't grow as much as the other side. A need exists for a device that can be used to make the smile more symmetric.

The present invention provides a cosmetic prosthesis which is easy and inexpensive to prepare, easy to install and remove, thus comfortably displacing the lips as to reduce facial wrinkles and ageing lines.

### SUMMARY OF THE INVENTION

The disadvantages described above are overcome and other advantages are achieved in a cosmetic prosthesis. The prosthesis includes a customizable space filling component which rest on the upper or lower gums against the under side of the lips pushing the lips outward to reduce facial wrinkles around the mouth. The prosthesis can be constructed to have a thickness selected to achieve a desired facial appearance.

In one embodiment, the cosmetic prosthesis is fitted to the gums through dental impression techniques. The cosmetic prosthesis can be constructed for attachment to the teeth for stability. It is preferred for the cosmetic prosthesis to be constructed to push or contour the lips outward.

It is also preferred for the cosmetic prosthesis components to be color matched to the patients gums.

In another embodiment, the cosmetic appliance or prosthesis is designed to provide non-invasive facial enhancement and is constructed to form first and second components each of which rest in a vestibule of the mouth. In various embodiments, the prosthetic can be formed from either elastomeric material, acrylic resins, flexible plastics or silicone compounds.

In a still further embodiment, the prosthetic includes a front side having a surface structure and color that simulates the natural gums and a reverse side adapted to be mounted on the gums. In such an embodiment, one longitudinal edge of said appliance has an indentation in a region adapted to contact or lie near the frenulum, the opposite longitudinal edge of the appliance is formed to be short of the tooth necks.

In a still further embodiment, the appliance contains titanium wire extending from the appliance or molded clasp, which holds the appliance to the teeth.

### BRIEF DESCRIPTION OF THE DRAWINGS

The foregoing summary, as well as the following detailed description of the preferred embodiments, is better understood when read in conjunction with the appended drawings. For the purpose of illustrating the invention, there are shown in the drawings embodiments that are presently preferred, it being understood, however, that the invention is not limited to the specific apparatus, system, and instrumentalities disclosed. In the drawings:

FIG. 1 is a perspective view of the invention before positioning on the lower gums;

FIG. 2 is a perspective view of the invention positioned on the lower gums;

FIG. 3 is a perspective view of the invention before positioning on the upper gums;

FIG. 4 is a perspective view of the invention positioned on the upper gums;

FIG. 5 is a top plan view of an alternative embodiment of the invention; and

FIG. 6 is an enlarged sectional view taken along the line 6-6 in FIG. 5.

### DETAILED DESCRIPTION OF ILLUSTRATIVE EMBODIMENTS

This invention relates to a cosmetic prosthesis for the mouth. The cosmetic prosthesis is useful to provide non-invasive enhancement of upper and/or lower lip contour for cosmetic purposes including the reduction or elimination of facial wrinkles and aging lines in the area. The prosthesis of the present invention, which functions like a swelling of the patients original gums, is constructed so that it can be custom fitted for each patient based on impression models. The prosthesis consists of one or two customizable space filling components which rest in the vestibule of the mouth underlying the upper lip and cheek. These space filling sub units or prosthesis rest on either side of the frenulum; the exact distance between the frenulum and these space filling components is variable depending upon the exact contour desired in a given patient.

The prosthesis can be made from inert elastomers and/or silicone compounds, resistant to chemical and mechanical influences in the mouth. If the prosthesis is constructed to form two sub units a wire can be used connecting these is made in the manner of an orthodontic retainer to provide easy insertion and removal.

The prosthetic can also be constructed from acrylic resin and/or flexible plastics, resistant to chemical and mechanical influences in the mouth. Again, if the prosthesis is constructed to form two sub units a wire connecting these is made in the manner of an orthodontic retainer to provide easy insertion and removal. When constructed of plastic, the wire is preferably replaced with a formed plastic clasp.

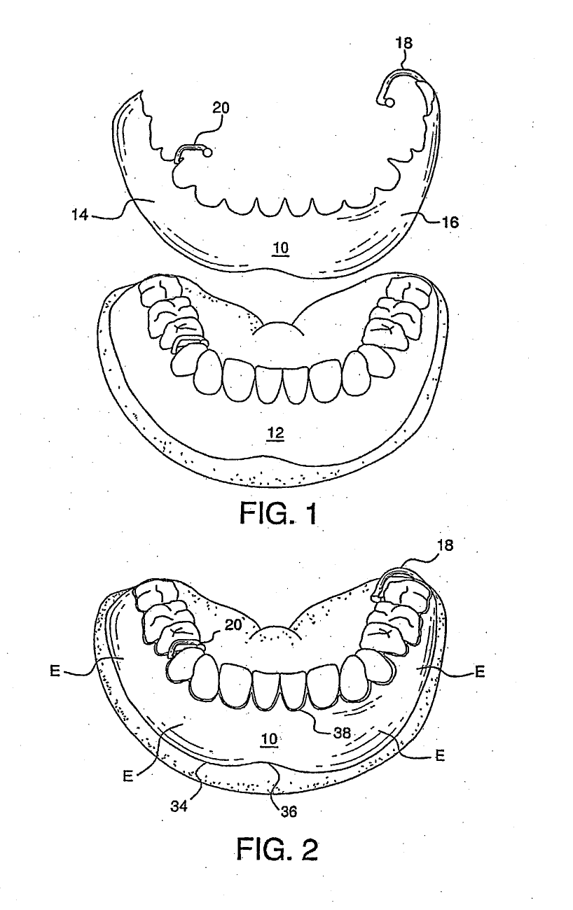

FIG. 1, shows a lower lip prosthesis 10 and a typical lower jaw 12. Prosthesis 10 has not yet been positioned or mounted to lower jaw 12. The prosthesis 10, which replicates on an inner surface the shape and image of the gums and replicates on the outer surface 14 and 16 the desired shape and image of the gums. Prosthesis 10 embodies an attachment wires 18 and 20 which connects to the teeth to hold prosthesis 10 in place.

FIG. 2 shows prosthesis 10 placed over the gums and attached to the teeth. In a preferred embodiment, prosthesis 10 is colored and shaped to replicate the desired shape of the gums displacing the existing space between the gums and the lips.

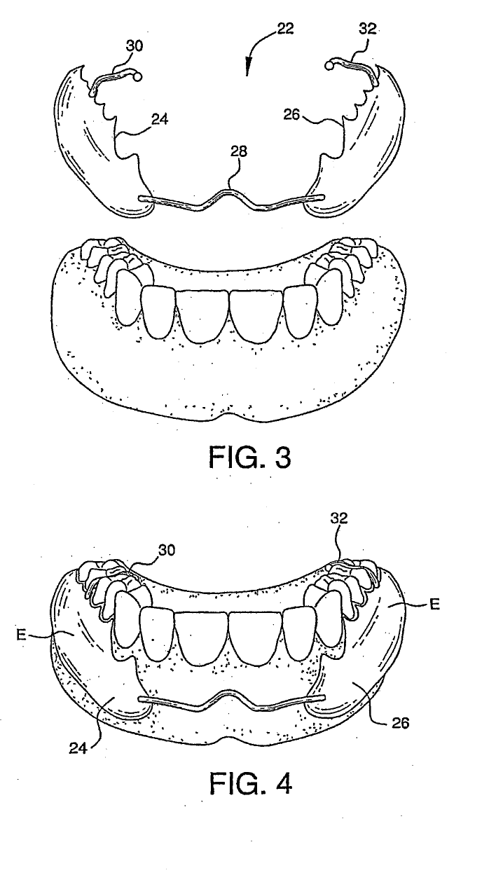

FIG. 3 shows an upper lip prosthesis 22. In this embodiment, prosthesis 22 includes two sub units 24 and 26. A connecting wire 28 holds the right (26) and left (24) sub units of the prosthesis together. The figure also illustrates the connecting wires 30 and 32 which helps hold the device firmly in place. When prosthesis 22 is formed from plastic, the connecting wires are replaced with formed plastic clasps.

FIG. 4, shows the prosthesis (22) in place over the gums of the upper lip area. The figure illustrates a clips 30 and 32 placed between teeth in the rear of the mouth.

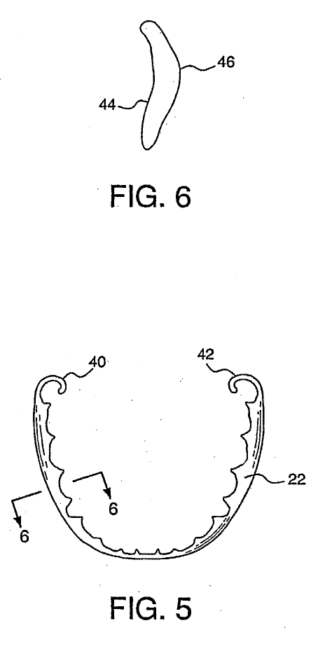

FIG. 5 depicts an embodiment in which prosthesis 22 is formed from plastic. In this embodiment, the connecting wires are replaced with formed plastic clasps 40, 42. FIG. 6 is a sectional view depicting prosthesis 22 in cross-section. Prosthesis 22 is shown to have an inner surface 44 and an outer surface 46. As will be appreciated from the description herein, inner surface 44 is preferable shaped to conform to the existing shape of the gum. The shape of surface 46 and its distance from surface 44, defines a distance or thickness profile. The shape of surface 46 together with a given thickness profile operates to reduce wrinkles and ageing lines.

The cosmetic appliance of the present invention overcomes in simple manner evasive techniques of surgical implants placed into the lip areas and under the nose to reduce wrinkles and ageing lines. In one embodiment of the invention, elastomers are used for construction of the appliance. In spite of their high Shore hardness, these elastomers are soft, highly elastic, inert, i.e. resistant to chemical and mechanical influences in the mouth, and physiologically harmless. As they are transparent and almost invisible on the gums and preferably are colored in any known manner to match the respective gums.

In another embodiment, and as shown in FIG. 4, the device is enlarged in region E above and just lateral to the oral commissural (corner of the mouth). Such a construction would help to elevate the corners of the mouth, and make and upward movement of the corners of the mouth.

Referring to FIGS. 2 and 4, devices 10 and 22 are shown to include a front side that preferably has a surface structure and color that simulates the natural gums and a reverse side adapted to be mounted on the gums. One longitudinal edge 34 device 10 has an indentation 36 in a region adapted to contact or lie near the frenulum, the opposite longitudinal edge 38 is short of the tooth necks.

The appliance may be removed from the mouth and cleaned effectively at any time desired. The appliance may be worn during eating or sleeping. An essential advantage of the appliance according to the invention must also be seen in the fact that it is inexpensive to prepare and does not require manipulations on healthy teeth for fitting or holding.

Again, the cosmetic appliance of the invention is safely held in place by the use of titanium or stainless steel wire or molded clasps which are carefully placed between or around molars.

In one embodiment, the permanently soft and pliable appliance of the present invention may be prepared as follows:

An impression of the upper and lower teeth and gum region of a patient is taken and a working model of plaster prepared from said impression. On the working model, areas of the gums which can most beneficially displace the lips are filled in with wax or a heat-stable material until the original state of the gums or some other desired thickness has been simulated. A mold for the cosmetic appliance is then prepared from the model thus obtained.

The elastomers used for preparing the cosmetic appliance, especially the heat-curable silicone rubbers, may be cured either by peroxidic cross-linking or by cross-linking under addition. Depending on the method of cross-linking employed, curing temperatures of between 100° C. and 180° C. are required. On account of these temperatures, the mold should be prepared from plaster or metals such as tin or metal alloys having a melting point above 200° C.

Silicone rubber worked into a dough-like consistency introduced into the mold and cured therein under pressure. The temperature and time needed for curing depend upon such factors as cross-linking agent used, type of mold and means of heating. For instance, in a plaster mold and under heating by means of a water bath, a method normally preferred in prosthetic dentistry, curing of the mask will take approximately 2 hours at 100° C. when 1 to 1.5% of bisbenzoylperoxide is used as a cross-linking agent. In metal molds needed for heat-curable silicone rubbers which are cross-linkable under addition, the appliance will cure within 5 to 15 minutes at a temperature of 170° C. to 180° C., the curing time depending on the thickness of the material layer.

Heat-curable silicone rubbers containing 1 to 1.5% of bisbenzoylperoxide may also be cured in metal molds. In this case, curing will take 5 to 15 minutes at temperatures of between 110° C. to 130° C.

Two-component rubbers cross-linkable under addition, especially those setting at room temperature, are admixed in equal parts and may be cured in either metal or plaster molds.

As the cosmetic appliance suitably should have the color of the natural gums, the basic material is admixed with a sufficient amount of a physiologically acceptable dye.

In yet a further embodiment of the invention, the prosthesis or the sub units may also be made as follows:

An impression of the upper and lower teeth and gum region of a patient is taken and a working model of plaster prepared from said impression. On the working model, areas of the gums which most beneficially displace the lips are filled in with wax or a heat-stable material until the original state of the gums has been simulated. A titanium clasp in now placed into the wax which will hold the device into place once placed into the patient's mouth, being careful not to effect the occlusion. A mold for the cosmetic appliance is then prepared from the model thus obtained.

The mold and wax adaptations are now placed into a sealed containment unit known as a capsule or flask. The capsule or flask is filled with investment (heat resistant material). The containment unit is heated to allow the wax to melt away from the mold and out of the unit. Now that the wax is out of the capsule and off of the mold, a resin (acrylic) is poured into the capsule filling the cavity the wax has left in the investment. The capsule is now placed into a pressure cooker and heated to the appropriate amount for the resin to harden. The pressure cooker is should operate at 24 PSI for the appropriate amount of time for the resin. The capsule will then be bench set to cool. Once removed from said capsule, the appliance will be trimmed and polished.

Flexible plastic material may be used as well for a stronger, softer and more flexible appliance. This method will differ slightly from the above described resin technique. A metal or titanium clasp is not necessary when using the plastic as the material can be formed to clasp. The plastic material is heated and must be hydraulically injected into the capsule or flask after the wax has been removed. This method eliminates the chance of bubbles in the appliance which weakens its integrity.

Silicone rubber worked into a dough-like consistency introduced into the mold and cured therein under pressure. The temperature and time needed for curing depend upon such factors as cross-linking agent used, type of mold and means of heating. For instance, in a plaster mold and under heating by means of a water bath, a method normally preferred in prosthetic dentistry, curing of the mask will take approximately 2 hours at 100° C. when 1 to 1.5% of bisbenzoylperoxide is used as a cross-linking agent. In metal molds needed for heat-curable silicone rubbers which are cross-linkable under addition, the appliance will cure within 5 to 15 minutes at a temperature of 170° C. to 180° C., the curing time depending on the thickness of the material layer.

Heat-curable silicone rubbers containing 1 to 1.5% of bisbenzoylperoxide may also be cured in metal molds. In this case, curing will take 5 to 15 minutes at temperatures of between 110° C. to 130° C.

Two-component rubbers cross-linkable under addition, especially those setting at room temperature, are admixed in equal parts and may be cured in either metal or plaster molds.

As the cosmetic appliance suitably should have the color of the natural gums, the basic material is admixed with a sufficient amount of a physiologically acceptable dye.

The cosmetic appliance of the invention differs from prior inventions relating to gum atrophy or paradentosis as the cosmetic appliance can be made from similar materials, however the purpose and/or function of the current invention is not to protect, cover or fit to exposed areas of the teeth beneath, above or below the gum line. The primary function of the device is to fill the cavity within the mouth between the gums and the lips to dissipate facial wrinkles.

The cosmetic appliance of the invention differs from prior inventions relating to dentures or similar as the current invention does not cover the top of the gums. The current invention does not cover, protect, maintain or house teeth, prosthetic teeth or similar.

The cosmetic appliance of the invention differs from prior inventions relating to orthodontics as the invention does not alter of affect the occlusion of the teeth. The current invention does not alter, adjust or touch the teeth other than the clasping device which hold the appliance in place.

A still further embodiment of the cosmetic prosthesis is safely held in place by a clamping device molded onto the prosthesis which temporarily attaches to the molars.

The cosmetic prosthesis of the invention differs from prior inventions relating to gum atrophy or paradentosis as the cosmetic prosthesis can be made from similar materials, however the purpose and/or function of the current invention is not to protect, cover or fit to exposed areas of the teeth beneath, above or below the gum line. The primary function of the device is to fill the cavity within the mouth between the gums and the lips to dissipate facial wrinkles.

In one embodiment of the invention, the prosthesis includes a front side having a surface structure and color that simulates the natural gums and a reverse side adapted to be mounted on the gums, one longitudinal edge of said appliance having an indentation in a region adapted to contact or lie near the frenulum, the opposite longitudinal edge of said appliance being short of the tooth necks.

It is to be understood that the foregoing illustrative embodiments have been provided merely for the purpose of explanation and are in no way to be construed as limiting of the invention. Words which have been used herein are words of description and illustration, rather than words of limitation. Further, although the invention has been described herein with reference to particular structure, materials and/or embodiments, the invention is not intended to be limited to the particulars disclosed herein. Rather, the invention extends to all functionally equivalent structures, methods and uses, such as are within the scope of the appended claims. Having the benefit of the teachings of this specification, others may affect numerous modifications thereto and changes may be made without departing from the scope and spirit of the invention in its aspects.

What is claimed:

1. A cosmetic prosthesis, comprising a customizable space filling component which rest on the upper or lower gums against the under side of the lips pushing the lips outward to reduce facial wrinkles around the mouth.

2. A cosmetic prosthesis, according to claim 1 wherein the prosthesis is constructed to have a thickness selected to achieve a desired facial appearance.

3. A cosmetic prosthesis, according to claim 1 wherein the prosthesis is fitted to the gums through dental impression techniques.

4. A cosmetic prosthesis, according to claim 1 wherein the prosthesis is attached to the teeth for stability.

5. A cosmetic prosthesis, according to claim 1, wherein the prosthesis is constructed to push or contour the lips outward.

6. A cosmetic prosthesis, according to claim 1 wherein the components are color matched to the patients gums.

7. A cosmetic appliance designed to provide non-invasive facial enhancement comprising first and second components each of which rest in a vestibule of the mouth.

8. The appliance of claim 7, wherein said appliance is formed from elastomeric material.

9. The appliance of claim 7, wherein said appliance is formed from acrylic resins.

10. The appliance of claim 7, wherein said appliance is formed from flexible plastics.

11. The appliance of claim 7, wherein said appliance is formed from silicone compounds.

12. The appliance of claim 7, wherein said appliance comprises a front side having a surface structure and color that simulates the natural gums and a reverse side adapted to be mounted on the gums, one longitudinal edge of said appliance having an indentation in a region adapted to contact or lie near the frenulum, the opposite longitudinal edge of said appliance being short of the tooth necks.

13. A cosmetic appliance according to claim 7 wherein the inert elastomeric or acrylic material is a thermosetting elastomer.

14. A cosmetic appliance according to claim 13 wherein the thermosetting elastomer or acrylic is a urethane elastomer.

15. A cosmetic appliance according to claim 13 wherein the thermosetting elastomer or acrylic is a heat-curable or a cold-setting two-component silicone rubber cross-linkable under addition.

16. A cosmetic appliance according to claim 7, wherein the appliance contains titanium wire extending from the appliance or molded clasp, which holds the appliance to the teeth.

17. A cosmetic appliance according to claim 7, wherein the appliance maintains a titanium wire connecting the left and right sides of said appliance

18. A cosmetic appliance according to claim 7, wherein the acrylic material is a thermosetting resin.

19. A cosmetic appliance according to claim 7 wherein the flexible plastic is of an injected nature.
